(19) United States
(12) Patent Application Publication
(10) Pub. No.: US 2019/0142504 A1
(43) Pub. Date: May 16, 2019

(54) TREATMENT TOOL

(71) Applicant: Olympus Corporation, Tokyo (JP)

(72) Inventor: Shoei Tsuruta, Tokyo (JP)

(73) Assignee: Olympus Corporation, Tokyo (JP)

(21) Appl. No.: 16/245,705

(22) Filed: Jan. 11, 2019

Related U.S. Application Data
(63) Continuation of application No. PCT/JP2016/071186, filed on Jul. 19, 2016.

(51) Int. Cl. A61B 18/14; A61B 18/12

(52) U.S. Cl. CPC A61B 18/1445; A61B 18/1482; A61B 18/1206

(57) ABSTRACT

A treatment tool includes a first grasping jaw having a first grasping surface. A second grasping jaw has a second grasping surface and is configured to engage with the first grasping jaw so as to pivot with respect to one another for holding a living tissue. A first electrode is disposed on the first grasping surface. A second electrode is disposed on one of the first grasping surface and in tandem with the first electrode supplies heat to the living tissue. A heat generator is disposed on at least one of the first grasping jaw and the second grasping jaw for generating heat when electrically energized. When the first grasping surface and the second grasping surface confront each other, the first electrode and the second electrode are disposed in respective positions on both sides of a central position of the heat generator.

### CROSS-REFERENCE TO RELATED APPLICATION

This application is a continuation application of PCT Application No. PCT/JP2016/071186 filed on Jul. 19, 2016, which is hereby incorporated by reference in its entirety.

### TECHNICAL FIELD

The present disclosure relates to a treatment tool.

### DESCRIPTION OF THE RELATED ART

Hereinbefore, there has been known a treatment tool for treating, e.g., joining or anastomosing, separating, or otherwise processing, a living tissue by applying energy to the living tissue as disclosed in Japanese Patent No. 3349139.

The treatment tool, or coagulation dissection forceps, disclosed in Japanese Patent No. 3349139 includes a first grasping jaw, or first grip, having a first grasping surface and a second grasping jaw, or second grip, having a second grasping surface for grasping a living tissue between itself and the first grasping surface. The first grasping jaw has a heat generator that, when electrically energized, generates heat to heat the first grasping surface. The treatment tool treats the living tissue by grasping the living tissue with the first and second grasping jaws and generating heat from the heat generator to heat the living tissue, i.e., by applying thermal energy to the living tissue.

For treating the living tissue using the thermal energy, the heat that is transferred to the living tissue is progressively spread from the heat source, i.e., the heat generator, at the center radially through the living tissue. Therefore, it takes time for the heat to be transferred thicknesswise, or along the direction in which the living tissue is grasped, across a treatment target tissue, grasped by the first and second grasping jaws, of the living tissue. It is thus difficult to shorten the time required to treat the living tissue. Furthermore, if sufficient time is spent until the heat is transferred thicknesswise across the treatment target tissue, then since the heat is progressively spread from the heat source, i.e., the heat generator, at the center radially through the living tissue, the thermal energy tends to act on a peripheral tissue that is present in the periphery of the treatment target tissue of the living tissue, possibly obstructing minimally invasive treatment of the treatment target tissue.

Accordingly, there is a need for a treatment tool that is capable of reducing treatment time and performing minimally invasive treatment.

### BRIEF SUMMARY OF EMBODIMENTS

In order to solve the problems described hereinbefore, a treatment tool in accordance with the present disclosure includes a first grasping jaw having a first grasping surface, a second grasping jaw having a second grasping surface for grasping a living tissue between itself and the first grasping surface, a first electrode disposed on the first grasping surface, a second electrode disposed on one of the first grasping surface and the second grasping surface and supplied with high-frequency electric power between itself and the first electrode, and a heat generator disposed on at least one of the first grasping jaw and the second grasping jaw and generating heat when electrically energized, in which when the first grasping surface and the second grasping surface confront each other, the first electrode and the second electrode are disposed in respective positions on both sides of a central position of the heat generator as viewed along directions in which the first grasping surface and the second grasping surface confront each other.

The treatment tool according to the present disclosure is advantageous in that it is capable of reducing treatment time and performing minimally invasive treatment.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrating a treatment system according to Embodiment 1 of the present disclosure.

FIG. 2 is a view illustrating a grasper illustrated in FIG. 1.

FIG. 3 is a view illustrating the grasper illustrated in FIG. 1.

FIG. 4 is a view illustrating the positional relationship between first and second electrodes and a thermal energy applying portion illustrated in FIGS. 2 and 3.

FIG. 5A is a view depicting an advantage of Embodiment 1 of the present disclosure.

FIG. 5B is a view depicting the advantage of Embodiment 1 of the present disclosure.

FIG. 5C is a view depicting the advantage of Embodiment 1 of the present disclosure.

FIG. 6 is a view illustrating a grasper of a treatment tool according to Embodiment 2 of the present disclosure.

FIG. 7 is a view illustrating a grasper of a treatment tool according to Embodiment 3 of the present disclosure.

FIG. 8 is a view illustrating a grasper of a treatment tool according to Embodiment 4 of the present disclosure.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Forms by which the present disclosure is embodied, hereinafter referred to as “embodiments,” will hereinafter be described with reference to the drawings. The embodiments to be described hereinafter should not be interpreted as limiting the present disclosure. Identical parts are denoted by identical numeral reference in figures.

### Embodiment 1

### Makeup Outline of a Treatment System

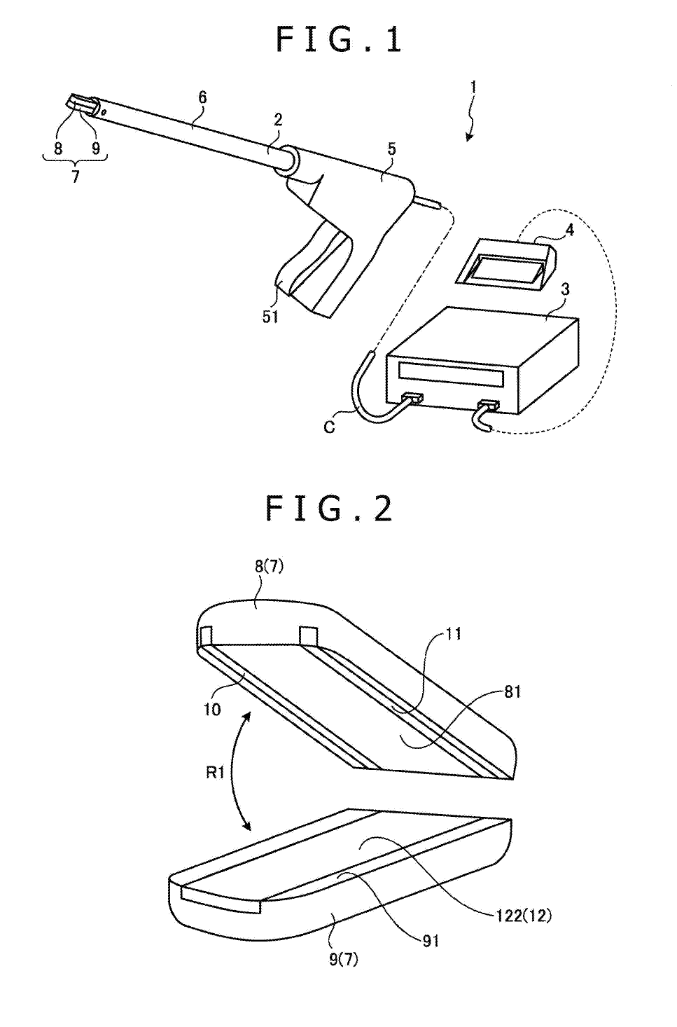

FIG. 1 is a view illustrating a treatment system 1 according to Embodiment 1 of the present disclosure.

The treatment system 1 treats, e.g., joins or anastomoses, separates, or otherwise processes, a living tissue by applying energy, e.g., thermal energy or electric energy (high-frequency energy), to the living tissue. As illustrated in FIG. 1, the treatment system 1 includes a treatment tool 2, a controller 3, and a foot switch 4.

Makeup of the Treatment Tool

The treatment tool 2 is a linear-type surgical treatment tool for treating a living tissue through an abdominal wall, for example. As illustrated in FIG. 1, the treatment tool 2 includes a handle 5, a sheath or shaft 6, and a grasper 7.

The handle 5 is a part by which the surgeon holds the treatment tool 2 by hand. As illustrated in FIG. 1, the handle 5 has a manipulating knob 51.

As illustrated in FIG. 1, the sheath or shaft 6 is of a substantially hollow cylindrical shape and has one end, i.e., a right end in FIG. 1, connected to the handle 5. The grasper 7 is mounted on the other end, i.e., a left end in FIG. 1, of the sheath or shaft 6. The sheath shaft 6 houses therein an opening and closing mechanism, not depicted, that opens and closes a pair of grasping jaws 8 and 9 (FIG. 1) that make up the grasper 7 in response to the surgeon's manipulation of the manipulating knob 51. An electric cable C (FIG. 1) connected to the controller 3 is housed in the shaft 6 and extends from one end, i.e., a right end in FIG. 1, to the other end, i.e., a left end in FIG. 1, through the handle 5.

Makeup of the Grasper

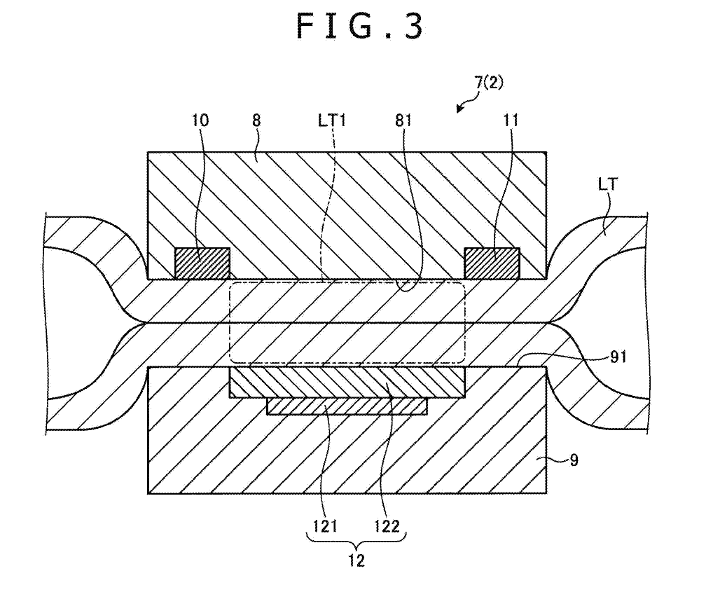

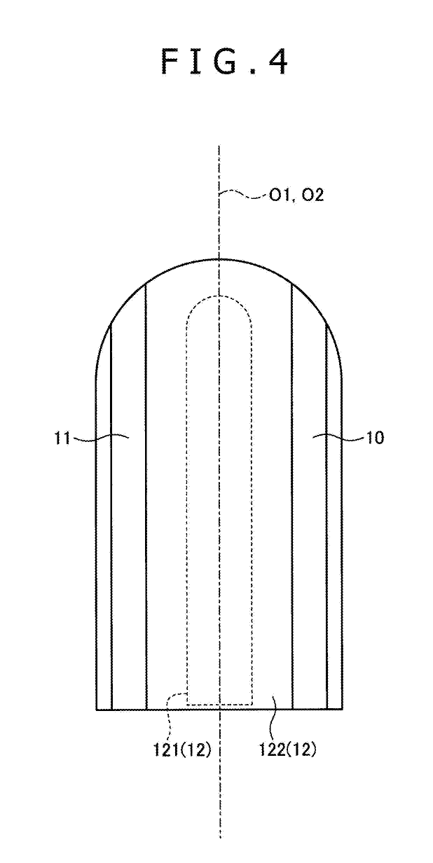

FIGS. 2 and 3 are views illustrating the grasper 7. Specifically, FIG. 2 is a perspective view illustrating the grasper 7 that is set to an open state, i.e., a state in which the pair of grasping jaws 8 and 9 are opened or spaced apart. FIG. 3 is a cross-sectional view of the grasper 7 that is set to a closed state grasping a living tissue LT, i.e., a state in which the pair of grasping jaws 8 and 9 are closed or a pair of grasping surfaces 81 and 91 confront each other, taken along a sectional plane along the widthwise directions of the grasper 7, i.e., the widthwise directions or leftward and rightward directions in FIGS. 2 and 3, perpendicular to the longitudinal directions interconnecting the distal and proximal ends of the grasper 7.

The grasper 7 is a portion that grasps the living tissue LT (FIG. 3) and treats the living tissue LT. As illustrated in FIGS. 1 through 3, the grasper 7 has the pair of grasping jaws 8 and 9.

The pair of grasping jaws 8 and 9 are pivotally supported on the other end of the shaft 6 such that the grasping jaws 8 and 9 can be opened and closed in the directions indicated by the arrow R1 (FIG. 2). The pair of grasping jaws 8 and 9 are able to grasp the living tissue LT in response to the surgeon's manipulation of the manipulating knob 51.

Makeup of the Grasping Jaws

Of the grasping jaws 8 and 9, the grasping jaw 8 is disposed above the other grasping jaw 9 in FIGS. 2 and 3, and is substantially shaped as a rectangular parallelepiped extending along the longitudinal directions interconnecting the distal and proximal ends of the grasping jaw 8. The grasping jaw 8 may be made of a material that is highly heat-resistant, low in thermal conductivity, and excellent in electric insulation, e.g., a resin such as PTFE (polytetrafluoroethylene), PEEK (polyetheretherketone), PBI (polybenzimidazole), or the like. However, the material of the grasping jaw 8 is not limited to the concerned resin, but may be ceramics such as alumina, zirconia, or the like. Furthermore, the grasping jaw 8 may be coated with PTFE, DLC (Diamond-Like Carbon), a ceramics-based insulative coating material, a silica-based insulative coating material, or a silicone-based insulative coating material that is nonadherent to living bodies.

The grasping jaw 8 has a lower surface in FIGS. 2 and 3 that functions as a grasping surface 81 for grasping the living tissue LT between itself and the other grasping jaw 9. The grasping surface 81 will hereinafter be referred to as “one grasping surface 81” in order to distinguish from a grasping surface 91, to be described hereinafter, of the other grasping jaw 9, whereas the grasping surface 91 as “other grasping surface 91.”

According to Embodiment 1, the one grasping surface 81 has a flat shape.

As illustrated in FIGS. 2 and 3, first and second electrodes 10 and 11 are embedded in the one grasping surface 81 at respective areas positioned on both end portions in the widthwise directions, i.e., on left and right end portions in FIGS. 2 and 3, and extending along the entire length, i.e., the entire length in the longitudinal directions, of the one grasping surface 81.

The first and second electrodes 10 and 11 are made of an electrically conductive material such as copper, aluminum, carbon, or the like, for example. Each of the first and second electrodes 10 and 11 is in the form of a plate substantially shaped as a rectangular parallelepiped extending along the longitudinal directions of the one grasping surface 81. The first and second electrodes 10 and 11 are embedded in the one grasping surface 81 such that one of the plate surfaces, i.e., the lower surface in FIGS. 2 and 3, of each of the first and second electrodes 10 and 11 makes up part of the one grasping surface 81, i.e., is exposed. The electric cable C, which extends from one end to the other end of the shaft 6, contains a pair of high-frequency leads, not depicted. The pair of high-frequency leads is connected respectively to the first and second electrodes 10 and 11. When the first and second electrodes 10 and 11 are supplied with high-frequency electric power from the controller 3 through the pair of high-frequency leads, the first and second electrodes 10 and 11 generate high-frequency energy. When the first and second electrodes 10 and 11 are supplied with high-frequency electric power while the grasping jaws 8 and 9, i.e., the grasping surfaces 81 and 91 thereof, are grasping the living tissue LT, a high-frequency potential is developed between the first and second electrodes 10 and 11, causing a high-frequency current to flow through the living tissue LT. In other words, the first and second electrodes 10 and 11 are a pair of electrodes where one of them functions as a positive electrode while the other as a negative electrode.

The first and second electrodes 10 and 11 are not limited to plates, but may be of a different shape such as round bars embedded in the grasping jaw 8 and having projected portions that are small as compared with the distance between the grasping jaws 8 and 9. The first and second electrodes 10 and 11 may not necessarily be made of a bulk material, but may be in the form of electrically conductive thin films of platinum or the like deposited by way of evaporation, sputtering, or the like. Moreover, the surfaces of the first and second electrodes 10 and 11 may not necessarily be physically exposed as described hereinbefore, but may be electrically exposed. Specifically, the surfaces of the first and second electrodes 10 and 11 may be coated with an electrically conductive coating material such as Ni-PTFE film, electrically conductive DLC (Diamond-Like Carbon) thin film, or the like that is nonadherent to living bodies, so that the surfaces can function as electrodes to develop a potential. Such alternatives do not depart from the scope of the present disclosure.

The other grasping jaw 9 is substantially shaped as a rectangular parallelepiped extending along the longitudinal directions interconnecting the distal and proximal ends of the grasping jaw 9. As with the one grasping jaw 8, the other grasping jaw 9 may be made of a resin such as PTFE, PEEK, PBI, or the like, or ceramics such as alumina, zirconia, or the like, for example.

The grasping jaw 9 has an upper surface in FIGS. 2 and 3 that functions as the other grasping surface 91 for grasping the living tissue LT between itself and the one grasping surface 81.

According to Embodiment 1, the other grasping surface 91 is formed flatwise as with the one grasping surface 81.

As illustrated in FIGS. 2 and 3, a thermal energy applying portion 12 is embedded in the other grasping surface 91 at an area positioned on a central portion in the widthwise directions, i.e., on a central portion in the left and right directions in FIGS. 2 and 3, and extending along the entire length of the other grasping surface 91.

As illustrated in FIGS. 2 and 3, the thermal energy applying portion 12 has a heat generator 121 (FIG. 3) and a heat transmitter 122.

The heat generator 121 is in the form of an electric resistance pattern having a substantially U shape. The U-shaped heat generator 121 extends at one leg in a longitudinal direction from the proximal end side (right side in FIG. 2) of the other grasping jaw 9 to the distal end side (left side in FIG. 2) thereof, is bent, and then extends at the other leg back toward the proximal end side. The electric cable C, which extends from one end to the other end of the shaft 6, contains a pair of heat-generating leads, not depicted, connected respectively to both ends of the heat generator 121. The heat generator 121 generates heat when the controller 3 applies a DC (Direct Current) or AC (Alternating Current) voltage thereto through the heat-generating leads, i.e., when the controller 3 electrically energizes the heat generator 121 through the heat-generating leads.

The heat generator 121 described hereinbefore is formed by machining an electrically conductive material of stainless steel (SUS304). The heat generator 121 is bonded by way of thermocompression bonding to a central portion in the widthwise directions of a lower surface in FIG. 3 of the heat transmitter 122.

The material of the heat generator 121 is not limited to stainless steel (SUS304), but may be other stainless steel materials such as those in 400s or electrically conductive materials including platinum, tungsten, etc. The heat generator 121 may not necessarily be bonded to the lower surface in FIG. 3 of the heat transmitter 122 by way of thermocompression bonding, but may be deposited on the lower surface in FIG. 3 of the heat transmitter 122 by way of evaporation, sputtering, etc.

The heat transmitter 122 is made of a composite material including a material that is highly heat-resistant, high in thermal conductivity, and excellent in electric insulation, e.g., a resin such as PTFE, PEEK, PBI, or the like, with ceramics or the like included therein as a thermally conductive filler, or a material including ceramics such as aluminum nitride or the like or an electrically conductive material such as copper, aluminum, carbon, or the like which is coated with an insulative material such as PTFE or the like. The heat transmitter 122 is in the form of a plate substantially shaped as a rectangular parallelepiped extending along the longitudinal directions of the other grasping surface 91. The heat transmitter 122 is embedded in the other grasping surface 91 such that the upper surface in FIGS. 2 and 3 of the heat transmitter 122 makes up part of the other grasping surface 91, i.e., is exposed. The heat transmitter 122 transmits heat from the heat generator 121 to the living tissue LT, i.e., applies thermal energy to the living tissue LT.

The heat transmitter 122 may include separate members having different transverse sizes, i.e., a lower heat transmitter member and an upper heat transmitter member, that are joined together for high thermal conduction in the directions along which the grasping jaws 8 and 9 are opened and closed, i.e., in the vertical directions in FIGS. 2 and 3. For example, a ceramic heater as the heat generator 121 may be deposited as an electrically conductive thin film on the lower heat transmitter member by sputtering, and the lower heat transmitter member may be bonded to the upper heat transmitter member by high thermal conduction such as nano-Ag particles or the like. In other words, the lower heat transmitter member and the upper heat transmitter member may be considered together as the heat transmitter 122.

The other grasping surface 91 as described hereinbefore may be coated with an insulative coating material described hereinbefore that is nonadherent to living bodies.

In Embodiment 1, the one grasping jaw 8 corresponds to a first grasping jaw according to the present disclosure. The one grasping surface 81 corresponds to a first grasping surface according to the present disclosure. The other grasping jaw 9 corresponds to a second grasping jaw according to the present disclosure. The other grasping surface 91 corresponds to a second grasping surface according to the present disclosure.

Positional relationship between the first and second electrodes and the thermal energy applying portion:

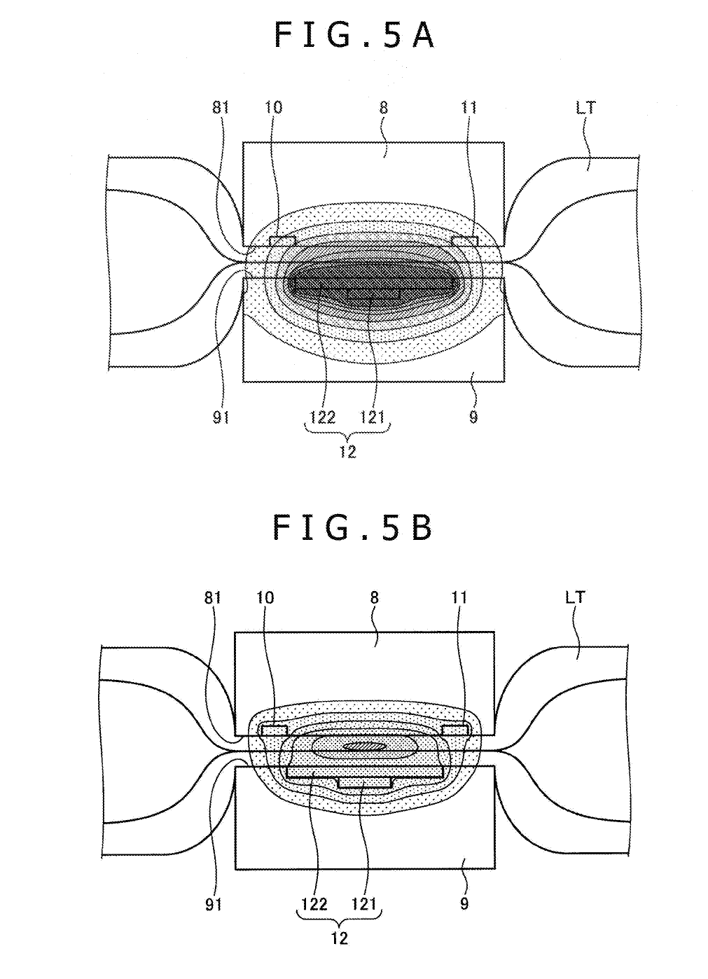

FIG. 4 is a view illustrating the positional relationship between the first and second electrodes 10 and 11 and the thermal energy applying portion 12. Specifically, FIG. 4 is a view illustrating the first and second electrodes 10 and 11 and the thermal energy applying portion 12 when the grasping jaws 8 and 9 are in the closed state, i.e., the grasping surfaces 81 and 91 confront each other, as viewed along the directions in which the grasping surfaces 81 and 91 confront each other, i.e., the directions normal to the grasping surfaces 81 and 91.

As illustrated in FIG. 4, the first and second electrodes 10 and 11 are disposed in respective positions on both sides of a central position O1 in the widthwise directions of the thermal energy applying portion 12, as viewed along the directions in which the grasping surfaces 81 and 91 confront each other in the closed state. Specifically, a central position O2 in the widthwise directions between the first and second electrodes 10 and 11 is aligned with the central position O1 of the thermal energy applying portion 12. Moreover, the first and second electrodes 10 and 11 are disposed outside of the heat generator 121 in the widthwise directions.

Makeup of the Controller and the Foot Switch

The foot switch 4 is a part that the surgeon operates with their foot. When the foot switch 4 is thus operated, the controller 3 selectively turns on and off the treatment tool 2, i.e., the first and second electrodes 10 and 11 and the heat generator 121.

Means for selectively turning on and off the treatment tool 2 is not limited to the foot switch 4, but may be a switch that can be operated by hand, etc.

The controller 3, which includes a CPU (Central Processing Unit) and so on, integrally controls operation of the treatment tool 2 according to predetermined control programs. Specifically, in response to the operation of the foot switch 4 by the surgeon to turn on the controller 3, the controller 3 supplies high-frequency electric power at a preset output level between the first and second electrodes 10 and 11 through the pair of high-frequency leads, and also applies electric power at a preset output level to the heat generator 121 through the pair of heat-generating leads at preset timings. Then, the controller 3 appropriately controls respective energy levels of the applied electric power.

Operation of the Treatment System

Next, operation of the treatment system 1 described hereinbefore will be described hereinafter.

The surgeon holds the treatment tool 2 by hand, and inserts a distal-end portion of the treatment tool 2, i.e., the grasper 7 and a portion of the shaft 6, into an abdominal cavity through the abdominal wall using a trocar or the like, for example. The surgeon also operates the manipulating knob 51 to grasp the living tissue LT with the pair of grasping jaws 8 and 9.

Then, the surgeon operates the foot switch 4 to turn on the controller 3 to electrically energize the treatment tool 2. When the controller 3 is turned on, the controller 3 supplies high-frequency electric power between the first and second electrodes 10 and 11 through the pair of high-frequency leads. When the high-frequency electric power is thus supplied between the first and second electrodes 10 and 11, a high-frequency current flows between the first and second electrodes 10 and 11, generating Joule heat in a treatment target tissue LT1 (FIG. 3) of the living tissue LT between the first and second electrodes 10 and 11. At the same time the controller 3 supplies the high-frequency electric power, the controller 3 applies electric power to the heat generator 121, i.e., electrically energizes the heat generator 121, through the pair of heat-generating leads. When the heat generator 121 is thus electrically energized, the heat generator 121 generates heat, which is transmitted through the heat transmitter 122 to the treatment target tissue LT1. The treatment target tissue LT1 is now treated by the Joule heat generated therein and the heat transmitted thereto from the heat transmitter 122.

The timing at which the high-frequency electric power is supplied between the first and second electrodes 10 and 11 and the timing at which the electric power is applied to the heat generator 121, i.e., the heat generator 121 is electrically energized, may not necessarily be the same as each other, but may be different from each other.

Embodiment 1 described hereinbefore offers the following advantages:

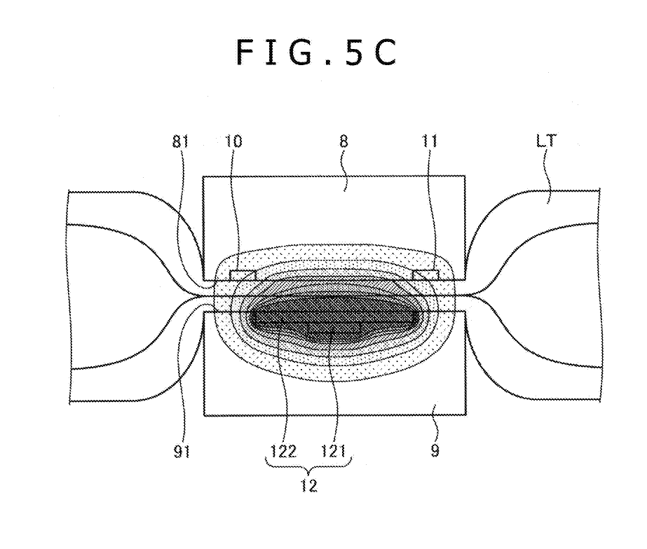

FIGS. 5A through 5C are views illustrative of the advantages of Embodiment 1 of the present disclosure. Specifically, FIG. 5A is a view illustrating the results of a simulation, and depicts a temperature distribution in case no high-frequency electric power is supplied between the first and second electrodes 10 and 11 and the heat generator 121 is maintained at a preset temperature, i.e., only thermal energy is applied to the living tissue LT. FIG. 5B is a view illustrating the results of a simulation, and depicts a temperature distribution in case the heat generator 121 generates no heat and high-frequency electric power at a preset output level is supplied between the first and second electrodes 10 and 11, i.e., only high-frequency energy is applied to the living tissue LT. FIG. 5C is a view illustrating the results of a simulation, and depicts a temperature distribution in case high-frequency electric power at a preset output level is supplied between the first and second electrodes 10 and 11 and electric power at a preset output level is applied to the heat generator 121, i.e., both thermal energy and high-frequency energy are applied to the living tissue LT.

In FIGS. 5A through 5C, blacker areas represent areas at higher temperatures, whereas whiter areas represent regions at lower temperatures. In other words, in FIGS. 5A through 5C, the temperatures of lighter areas are lower, whereas the temperatures of darker areas are higher.

FIGS. 5A through 5C also illustrate the temperature distributions at the instant the one grasping surface 81 that is devoid of the thermal energy applying portion 12 has reached a desired temperature of approximately 200° C.

When the living tissue LT is treated using thermal energy, the heat transmitted to the living tissue LT is progressively spread radially from the heat source, or the heat generator 121, at the center depending on the thermal conductivity of the materials involved. Therefore, the treatment is problematic in that it takes time until the heat is transmitted thicknesswise across the treatment target tissue LT1, i.e., in the directions along which the living tissue LT is grasped or in the vertical directions in FIGS. 3, and 5A through 5C.

According to the results of the simulation in which only “thermal energy is applied to the living tissue LT,” a time of T1 seconds, i.e., approximately 2.4 seconds, is consumed until the central portion in the widthwise directions of the one grasping surface 81 reaches a desired temperature of approximately 200° C. after the thermal energy has started to be applied (FIG. 5A). The results of the simulation also indicate that in addition to the fact that after the time of T1 seconds, the treatment target tissue LT1 is continuously held in contact with the thermal energy applying portion 12 for the time of T1 seconds, a peripheral tissue that is present in the periphery of the treatment target tissue LT1 is also kept at a relatively high temperature (FIG. 5A).

When the living tissue LT is treated using high-frequency energy, the high-frequency current flows between the first and second electrodes 10 and 11. With the first and second electrodes 10 and 11 being disposed in respective positions one another along the widthwise directions of the one grasping surface 81 according to Embodiment 1, the high-frequency current flows in the widthwise directions of the grasping jaws 8 and 9, i.e., in the leftward and rightward directions in FIGS. 3 and 5A through 5C. Since the area through which the high-frequency current flows between the first and second electrodes 10 and 11 is an area where heat is generated, the treatment target tissue TL1 is limited to a region close to the center in the widthwise directions of the grasping jaws 8 and 9, i.e., between the first and second electrodes 10 and 11. The temperature is highest in the area of the treatment target tissue LT1 which is spaced from the grasping surfaces 81 and 91 in the thicknesswise directions thereof. However, as the high-frequency energy is applied, the treatment target tissue LT1 starts to dry or dehydrate and has its impedance increased, resulting in a reduction in the action of high-frequency energy from a certain time on, so that the living tissue LT may not be treated as desired. The “certain time” referred to may be, for example, the “time when the power supply capacity fails to catch up with the increased impedance” or the “time when the amount of generated heat does not exceed the amount of heat lost by way of heat dissipation or heat transfer and fails to contribute to an increase in the temperature of the living tissue LT.”

According to the results of the simulation in which “only high-frequency energy is applied to the living tissue LT,” the treatment target tissue TL1 is limited to the region close to the center in the widthwise directions of the grasping jaws 8 and 9 and the temperature is highest in the area of the treatment target tissue LT1 which is spaced from the grasping surfaces 81 and 91 in the thicknesswise directions thereof (FIG. 5B). However, even upon elapse of the time of T1 seconds, i.e., approximately 2.4 seconds, after the high-frequency energy has started to be applied, the highest temperature attained by the treatment target tissue LT1 does not reach a desired temperature of approximately 200° C., e.g., the highest attainable temperature is approximately 150° C. (FIG. 5B).

When the living tissue LT is treated using both thermal energy and high-frequency energy according to Embodiment 1, the problems described hereinbefore can be solved as indicated by the results of the simulation (FIG. 5C) in which both “thermal energy and high-frequency energy are applied to the living tissue LT.”

Specifically, when the high-frequency energy is applied, it has an assistive effect on the thermal energy that is applied. The time that is consumed until the center in the widthwise directions of the one grasping surface 81 reaches a desired temperature of approximately 200° C. after both thermal energy and high-frequency energy have started to be applied is a time of T2 seconds, i.e., approximately 1.5 seconds, that is nearly 60 percent shorter than the time of T1 seconds consumed when “only thermal energy is applied to the living tissue LT.” The results of the simulation also indicate that since the time of T2 seconds is shorter, in addition to the fact that the treatment target tissue LT1 needs to be continuously held in contact with the thermal energy applying portion 12 for only the time of T2 seconds, the effect that the heat has on the peripheral tissue that is present in the periphery of the treatment target tissue LT1 is reduced, and the peripheral tissue is of a relatively low temperature.

The treatment tool 2 according to Embodiment 1 is therefore advantageous in that the treatment time is shortened and the treatment target tissue LT1 may be treated minimally invasively. Furthermore, as the total amount of heat that the material of the grasping jaws 8 and 9 receives is small, the treatment tool 2 is also advantageous in that the temperature of the grasping jaws 8 and 9 is lowered after the treatment is finished.

It is known that the highest attainable temperature of the living tissue LT can be increased depending on the living tissue LT and the conditions of the energy applied. The attainable temperature that is required for the heat generator 121 can be lowered, resulting in a contribution to the increased reliability of the heat generator 121.

With the treatment tool 2 according to Embodiment 1, in particular, the first and second electrodes 10 and 11 are disposed in the respective positions on both sides of the central position O1 in the widthwise directions of the heat generator 121, as viewed along the directions in which the grasping surfaces 81 and 91 confront each other. Specifically, the central position O2 in the widthwise directions between the first and second electrodes 10 and 11 is aligned with the central position O1. In other words, the area where heat is generated by the applied high-frequency energy and the area where heat is generated by the applied thermal energy are superposed one on the other. Therefore, the assistive effect referred to hereinbefore is enhanced, and the advantages that “the treatment time is shortened and the treatment target tissue LT1 may be treated minimally invasively” as described hereinbefore can appropriately be achieved.

With the treatment tool 2 according to Embodiment 1, moreover, the first and second electrodes 10 and 11 are disposed outside of the heat generator 121 in the widthwise directions, as viewed along the directions in which the grasping surfaces 81 and 91 confront each other. Stated otherwise, the heat generator 121 is disposed centrally in the widthwise directions in the other grasping surface 91. Consequently, the effect that the thermal energy has on the peripheral tissue that is present in the periphery of the treatment target tissue LT1 is further reduced.

If the first and second electrodes 10 and 11 and the thermal energy applying portion 12 are disposed on one of the grasping jaws 8 and 9, i.e., on the same grasping jaw, then the following problems are likely to occur:

When the thermal energy applying portion 12 applies thermal energy to the living tissue LT, the tissue that is present in the periphery of the first and second electrodes 10 and 11 dries or dehydrates, and has its impedance increased, resulting in a reduction in the action of high-frequency energy by the first and second electrodes 10 and 11. In other words, the assistive effect referred to hereinbefore is reduced.

With the treatment tool 2 according to Embodiment 1, the first and second electrodes 10 and 11 and the thermal energy applying portion 12 are disposed on the different grasping jaws. Therefore, the problems described hereinbefore are not likely to occur. Specifically, inasmuch as high-frequency electric power tends to avoid the tissue whose impedance has been increased by the action of the thermal energy applying portion 12, and to be selectively applied to the untreated living tissue LT that is of a low impedance, the high-frequency electric power is capable of more effectively assisting the treatment.

### Embodiment 2

Next, Embodiment 2 of the present disclosure will be described hereinafter.

In the description of Embodiment 2, the structural details which are similar to those of Embodiment 1 described hereinbefore are denoted by identical numeral reference and will not be described in detail or will be described briefly.

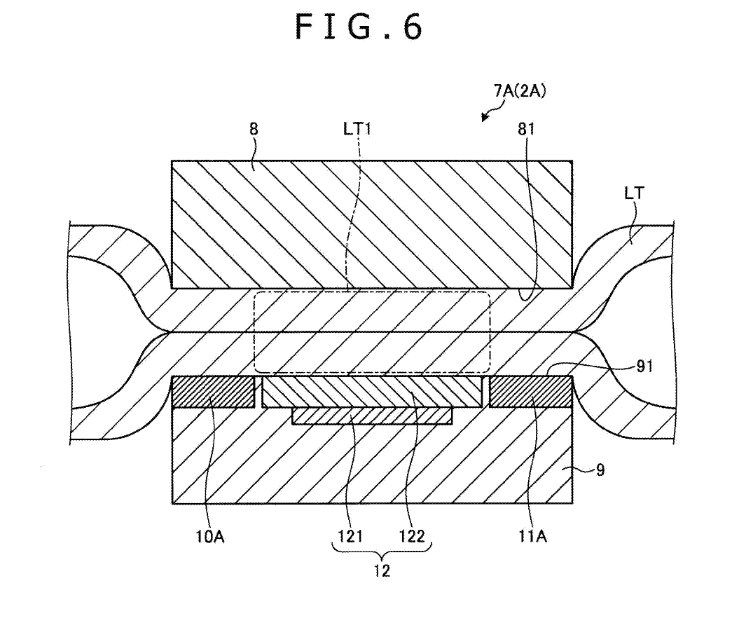

FIG. 6 is a view illustrating a grasper 7A of a treatment tool 2A according to Embodiment 2 of the present disclosure. Specifically, FIG. 6 is a cross-sectional view corresponding to FIG. 3.

As illustrated in FIG. 6, the treatment tool 2A according to Embodiment 2 is different from the treatment tool 2 (FIG. 3) described hereinbefore in Embodiment 1 as to the positions where the first and second electrodes according to the present disclosure are disposed.

As illustrated in FIG. 6, the one grasping surface 81 of the grasping jaw 8 according to Embodiment 2 is devoid of the first and second electrodes 10 and 11. Though the one grasping surface 81 according to Embodiment 2 is devoid of the first and second electrodes 10 and 11, it has a flat shape as with Embodiment 1 described hereinbefore. The one grasping surface 81 may be coated with an insulative coating material that is nonadherent to living bodies as with Embodiment 1 described hereinbefore.

As illustrated in FIG. 6, the other grasping jaw 9 according to Embodiment 2 has first and second electrodes 10A and 11A in addition to the thermal energy applying portion 12 in the other grasping surface 91 thereof.

The first and second electrodes 10A and 11A have the same shape and function, i.e., the function to apply high-frequency energy to the living tissue LT, i.e., the treatment target tissue LT1, as the first and second electrodes 10 and 11 described hereinbefore with respect to Embodiment 1.

The first and second electrodes 10A and 11A are embedded in the other grasping surface 91 at respective areas positioned on both end portions in the widthwise directions, i.e., on both sides of the thermal energy applying portion 12, and extending along the entire length of the other grasping surface 91. The first and second electrodes 10A and 11A make up part of the other grasping surface 91. Though the other grasping surface 91 according to Embodiment 2 has the first and second electrodes 10A and 11A embedded therein, the other grasping surface 91 has a flat shape as with Embodiment 1 described hereinbefore. Areas of the other grasping surface 91 which are defined by upper surfaces in FIG. 6 of the first and second electrodes 10A and 11A may be coated with an electrically conductive coating material that is nonadherent to living bodies as with Embodiment 1 described hereinbefore, and the other area of the other grasping surface 91, i.e., the area defined by an upper surface in FIG. 6 of the heat transmitter 122, may be coated with an insulative coating material that is nonadherent to living bodies as with Embodiment 1 described hereinbefore.

In Embodiment 2, the positional relationship of the first and second electrodes 10A and 11A and the thermal energy applying portion 12 as viewed along the directions in which the grasping surfaces 81 and 91 confront each other in the closed state is similar to their positional relationship in Embodiment 1 described hereinbefore.

The first and second electrodes 10A and 11A are not limited to plates, but may be of a different shape such as round bars embedded in the grasping jaw 9 and having projected portions that are small as compared with the distance between the grasping jaws 8 and 9. The first and second electrodes 10A and 11A may not necessarily be made of a bulk material, but may be in the form of electrically conductive thin films of platinum or the like deposited by way of evaporation, sputtering, or the like.

In Embodiment 2, the other grasping jaw 9 corresponds to a first grasping jaw according to the present disclosure. The other grasping surface 91 corresponds to a first grasping surface according to the present disclosure. The one grasping jaw 8 corresponds to a second grasping jaw according to the present disclosure. The one grasping surface 81 corresponds to a second grasping surface according to the present disclosure.

As described hereinbefore, the treatment tool 2A according to Embodiment 2 offers similar advantages to those of Embodiment 1 described hereinbefore.

With the treatment tool 2A according to Embodiment 2, moreover, the other grasping jaw 9 has the first and second electrodes 10A and 11A and the thermal energy applying portion 12. Stated otherwise, the one grasping jaw 8 is free of any of the first and second electrodes 10A and 11A and the thermal energy applying portion 12. Therefore, the one grasping jaw 8 may be made simpler in structure and may be made smaller in size, i.e., the grasper 7A may be made smaller in diameter.

### Embodiment 3

Next, Embodiment 3 of the present disclosure will be described hereinafter.

In the description of Embodiment 3, the structural details which are similar to those of Embodiment 1 described hereinbefore are denoted by identical numeral reference and will not be described in detail or will be described briefly.

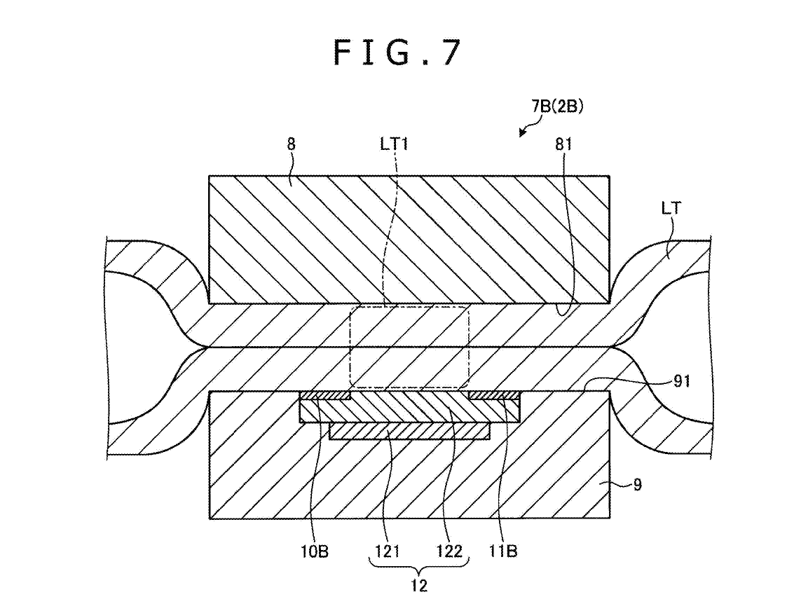

FIG. 7 is a view illustrating a grasper 7B of a treatment tool 2B according to Embodiment 3 of the present disclosure. Specifically, FIG. 7 is a cross-sectional view corresponding to FIG. 3.

As illustrated in FIG. 7, the treatment tool 2B according to Embodiment 3 is different from the treatment tool 2 (FIG. 3) described hereinbefore in Embodiment 1 as to the positions where the first and second electrodes according to the present disclosure are disposed and the process by which they are formed.

As illustrated in FIG. 7, the one grasping surface 81 of the grasping jaw 8 according to Embodiment 3 is devoid of the first and second electrodes 10 and 11 and has a flat shape as with Embodiment 2 described hereinbefore. The one grasping surface 81 may be coated with an insulative coating material that is nonadherent to living bodies as with Embodiment 1 described hereinbefore.

As illustrated in FIG. 7, the other grasping jaw 9 according to Embodiment 3 has first and second electrodes 10B and 11B in addition to the thermal energy applying portion 12 in the other grasping surface 91 thereof.

The first and second electrodes 10B and 11B have the same function, i.e., the function to apply high-frequency energy to the living tissue LT, i.e., the treatment target tissue LT1, as the first and second electrodes 10 and 11 described hereinbefore with respect to Embodiment 1, but are different therefrom as to the positions where the first and second electrodes 10B and 11B are disposed and the process by which they are formed.

Specifically, the first and second electrodes 10B and 11B are in the form of electrically conductive thin films of platinum or the like deposited by way of evaporation, sputtering, or the like. As illustrated in FIG. 7, the first and second electrodes 10B and 11B are disposed in respective areas positioned on both end portions in the widthwise directions of an upper surface in FIG. 7 of the heat transmitter 122 and extending along the entire length of the heat transmitter 122. The first and second electrodes 10B and 11B make up part of the other grasping surface 91. Though the other grasping surface 91 according to Embodiment 3 has the first and second electrodes 10B and 11B formed therein, the other grasping surface 91 has a flat shape as with Embodiment 1 described hereinbefore. Areas of the other grasping surface 91 which are defined by the first and second electrodes 10B and 11B may be coated with an electrically conductive coating material that is nonadherent to living bodies as with Embodiment 1 described hereinbefore, and the other area of the other grasping surface 91 may be coated with an insulative coating material that is nonadherent to living bodies as with Embodiment 1 described hereinbefore.

Specifically, according to Embodiment 3, since the first and second electrodes 10B and 11B are disposed in the heat transmitter 122, they are disposed in respective positions that are spaced from the outer edges in the widthwise directions of the other grasping surface 91. According to Embodiment 3, a central position in the widthwise directions between the first and second electrodes 10B and 11B and a central position in the widthwise directions of the thermal energy applying portion 12 are aligned with each other, as viewed along the directions in which the grasping surfaces 81 and 91 confront each other in the closed state, as with Embodiment 1 described hereinbefore.

The first and second electrodes 10B and 11B may not necessarily be in the form of thin films, but may be made of a bulk material, as with the first and second electrodes 10 and 11 according to Embodiment 1 described hereinbefore.

In Embodiment 3, the other grasping jaw 9 corresponds to a first grasping jaw according to the present disclosure. The other grasping surface 91 corresponds to a first grasping surface according to the present disclosure. The one grasping jaw 8 corresponds to a second grasping jaw according to the present disclosure. The one grasping surface 81 corresponds to a second grasping surface according to the present disclosure.

The treatment tool 2B according to Embodiment 3 offers similar advantages to those of Embodiments 1 and 2 described hereinbefore.

With the treatment tool 2B according to Embodiment 3, furthermore, the first and second electrodes 10B and 11B are disposed in the respective positions that are spaced from the outer edges in the widthwise directions of the other grasping surface 91. In other words, the dimension by which the first and second electrodes 10B and 11B are spaced from each other in the widthwise directions is reduced, thereby the treatment target tissue LT1 positioned between the first and second electrodes 10B and 11B is further limited to a region close to the center in the widthwise directions of the grasping jaws 8 and 9. Consequently, the effect that the heat has on the peripheral tissue that is present in the periphery of the treatment target tissue LT1 is further reduced.

The high-frequency current flowing between the first and second electrodes 10B and 11B changes its path over time as the impedance of the living tissue LT is increased by the high-frequency energy applied thereto. Specifically, in case the first and second electrodes 10B and 11B are disposed in the other grasping surface 91 according to Embodiment 3, a high-frequency current flows along a path close to the other grasping surface 91 immediately after high-frequency energy has started to be applied. As time goes by after the high-frequency energy has started to be applied, a high-frequency current flows along a path close to the one grasping surface 81. In other words, the path of the high-frequency current changes along the thicknesswise directions of the treatment target tissue LT1 over time.

Particularly in case the dimension by which the first and second electrodes 10B and 11B are spaced from each other in the widthwise directions is reduced according to Embodiment 3, the path of the high-frequency current changes in a relatively short period of time along the thicknesswise directions of the treatment target tissue LT1. Therefore, the assistive effect referred to hereinbefore is further increased, and the advantages that “the treatment time is shortened and the treatment target tissue LT1 may be treated minimally invasively” as described hereinbefore can appropriately be achieved.

### Embodiment 4

Next, Embodiment 4 of the present disclosure will be described hereinafter.

In the description of Embodiment 4, the structural details which are similar to those of Embodiment 1 described hereinbefore are denoted by identical numeral reference and will not be described in detail or will be described briefly.

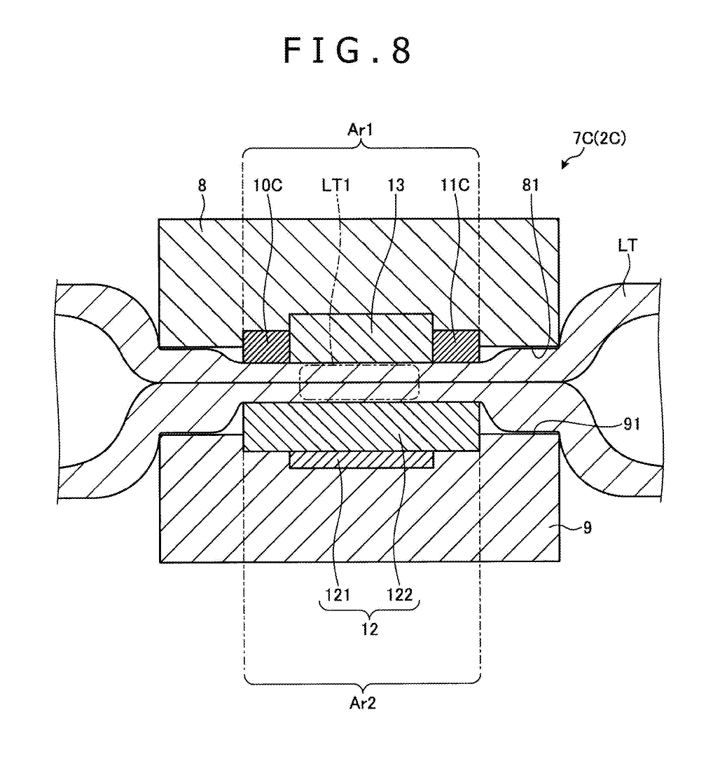

FIG. 8 is a view illustrating a grasper 7C of a treatment tool 2C according to Embodiment 4 of the present disclosure. Specifically, FIG. 8 is a cross-sectional view corresponding to FIG. 3.

As illustrated in FIG. 8, the treatment tool 2C according to Embodiment 4 is different from the treatment tool 2 (FIG. 3) described hereinbefore in Embodiment 1 as to the shape of the first and second grasping surfaces according to the present disclosure.

As illustrated in FIG. 8, the one grasping surface 81 of the one grasping jaw 8 according to Embodiment 4 has first and second electrodes 10C and 11C and an insulative member 13 disposed thereon.

The first and second electrodes 10C and 11C have the same function, i.e., the function to apply high-frequency energy to the living tissue LT, i.e., the treatment target tissue LT1, as the first and second electrodes 10 and 11 described hereinbefore with respect to Embodiment 1, but are different therefrom as to the positions where the first and second electrodes 10C and 11C are disposed.

Specifically, as illustrated in FIG. 8, the first and second electrodes 10C and 11C are embedded in respective areas of the one grasping surface 81 which are disposed in positions that are spaced from the outer edges in the widthwise directions of the one grasping surface 81 and that extend along the entire length of the one grasping surface 81. The first and second electrodes 10C and 11C are embedded such that they protrude from the one grasping jaw 8 toward the other grasping jaw 9. The first and second electrodes 10C and 11C have respective lower surfaces in FIG. 8 that define the one grasping surface 81.

The insulative member 13 is made of a material that is highly heat-resistant, low in thermal conductivity, and excellent in electric insulation, e.g., a resin such as PTFE, PEEK, PBI, or the like, or ceramics such as alumina, zirconia, or the like. The insulative member 13 is in the form of a plate substantially shaped as a rectangular parallelepiped extending along the longitudinal directions of the one grasping surface 81. The insulative member 13 is embedded in an area positioned between the first and second electrodes 10C and 11C in the one grasping surface 81 and extending along the entire length of the one grasping surface 81. Furthermore, the insulative member 13 is embedded such that it has a lower surface in FIG. 8 that lies flush with the lower surfaces in FIG. 8 of the first and second electrodes 10C and 11C and it protrudes from the one grasping jaw 8 toward the other grasping jaw 9. The lower surface in FIG. 8 of the insulative member 13 defines the one grasping surface 81.

The one grasping surface 81 according to Embodiment 4 has a projected shape in which a first central area Ar1 (FIG. 8) that is positioned centrally in the widthwise directions and defined by the respective lower surfaces in FIG. 8 of the first and second electrodes 10C and 11C and the insulative member 13 protrudes toward the other grasping jaw 9. Areas of the one grasping surface 81 which are defined by the first and second electrodes 10C and 11C may be coated with an electrically conductive coating material that is nonadherent to living bodies as with Embodiment 1 described hereinbefore, and the other area of the one grasping surface 81 may be coated with an insulative coating material that is nonadherent to living bodies as with Embodiment 1 described hereinbefore.

The first and second electrodes 10C and 11C may not necessarily be made of a bulk material, but may be in the form of electrically conductive thin films of platinum or the like deposited by way of evaporation, sputtering, or the like.

As illustrated in FIG. 8, the thermal energy applying portion 12 according to Embodiment 4 is embedded such that it protrudes from the other grasping jaw 9 toward the one grasping jaw 8.

The other grasping surface 91 according to Embodiment 4 has a projected shape in which a second central area Ar2 (FIG. 8) that is positioned centrally in the widthwise directions and defined by the upper surface in FIG. 8 of the heat transmitter 122 protrudes toward the one grasping jaw 8. The other grasping surface 91 may be coated with an insulative coating material that is nonadherent to living bodies as with Embodiment 1 described hereinbefore.

The first and second central areas Ar1 and Ar2 described hereinbefore have the same planar shape and confront each other in the closed state.

According to Embodiment 4, a central position in the widthwise directions between the first and second electrodes 10C and 11C and a central position in the widthwise directions of the thermal energy applying portion 12 are aligned with each other, as viewed along the directions in which the grasping surfaces 81 and 91 confront each other in the closed state, as with Embodiment 1 described hereinbefore.

In Embodiment 4, the one grasping jaw 8 corresponds to a first grasping jaw according to the present disclosure. The one grasping surface 81 corresponds to a first grasping surface according to the present disclosure. The other grasping jaw 9 corresponds to a second grasping jaw according to the present disclosure. The other grasping surface 91 corresponds to a second grasping surface according to the present disclosure.

The treatment tool 2C according to Embodiment 4 offers similar advantages to those of Embodiment 1 described hereinbefore.

With the treatment tool 2C according to Embodiment 4, moreover, the grasping surfaces 81 and 91 have the first and second central areas Ar1 and Ar2, respectively, and have the projected shapes, respectively. The first and second electrodes 10C and 11C are disposed in the first central area Ar1. The thermal energy applying portion 12 is disposed in the second central area Ar2. Therefore, the treatment tool 2C can compress the treatment target tissue LT1 under a high pressure to treat, e.g., join, anastomose, or seal, the treatment target tissue LT1 effectively.

### Other Embodiments

While the embodiments of the present disclosure have been described thus far, the present disclosure should not be limited to Embodiments 1 through 4 described hereinbefore.

In Embodiments 1 through 4 described hereinbefore, the grasping surfaces 81 and 91 are of a flat shape or a projected shape. However, the grasping surfaces 81 and 91 are not limited to those shapes, but may be of other shapes. For example, in order to make an incision in the living tissue LT effectively, at least one of the grasping surfaces 81 and 91 may be of a V-shaped cross section in which the portion of the grasping surface that corresponds to the position where the incision is to be made is in close proximity to the other grasping surface.

According to Embodiments 1 through 4 described hereinbefore, in order to apply high-frequency energy, the treatment tool has two electrodes, i.e., the first electrode 10 (10A to 10C) and the second electrode 11 (11A to 11C). However, the number of electrodes is not limited to two, but may be three or more. In order to apply thermal energy, the treatment tool has only one thermal energy applying portion 12. However, the treatment tool may have thermal energy applying portions 12 respectively in the grasping jaws 8 and 9.

In Embodiments 1 through 4 described hereinbefore, the positions where the first electrode 10 (10A to 10C), the second electrode 11 (11A to 11C), and the thermal energy applying portion 12 are disposed are not limited to the positions described in Embodiments 1 through 4 described hereinbefore. They may be disposed in other positions insofar as the first electrode 10 (10A to 10C) and the second electrode 11 (11A to 11C) are disposed in respective positions on both sides of the central position O1 in the widthwise directions of the thermal energy applying portion 12, as viewed along the directions in which the grasping surfaces 81 and 91 confront each other. For example, though the first electrode 10 (10A to 10C) and the second electrode 11 (11A to 11C) are disposed on one of the grasping surfaces 81 and 91 or on the same grasping surface according to Embodiments 1 through 4 described hereinbefore, they may be disposed on different grasping surfaces.

In Embodiment 4 described hereinbefore, both of the grasping surfaces 81 and 91 are of a projected shape. However, the grasping surfaces 81 and 91 are not limited to a projected shape, but, for example, one of the grasping surfaces 81 and 91 may be of a flat shape whereas the other may be of a projected shape, or one of the grasping surfaces 81 and 91 may be of a projected shape whereas the other may be of a recessed shape.

In Embodiments 1 through 4 described hereinbefore, the treatment tool 2 (2A to 2C) is arranged to treat the living tissue LT by applying thermal energy and high-frequency energy thereto. However, the treatment tool 2 (2A to 2C) is not limited to such an arrangement, but may be arranged to treat the living tissue LT by applying ultrasonic energy and optical energy such as laser or the like, in addition to thermal energy and high-frequency energy, thereto.

In sum, the disclosed technology is directed to A treatment tool comprises a first grasping jaw having a first grasping surface. A second grasping jaw having a second grasping surface and is configured to engage with the first grasping jaw so as to relatively pivot with respect to one another for holding a living tissue therebetween. A first electrode is disposed on the first grasping surface. A second electrode is disposed on either the first grasping surface or the second grasping surface and in tandem with the first electrode generates high-frequency energy to the living tissue held therebetween. A heat generator is disposed in at least one of the first grasping jaw and the second grasping jaw for generating heat when electrically energized. When the first grasping surface and the second grasping surface confront each other, the first electrode and the second electrode are configured to be disposed in respective positions on both sides of a central position of the heat generator as viewed along directions in which the first grasping surface and the second grasping surface confront each other.

When the first grasping surface and the second grasping surface confront one another, the respective first electrode and the second electrode are disposed outside of the heat generator in widthwise directions of the first grasping surface and the second grasping surface as viewed along the directions in which the first grasping surface and the second grasping surface confront one another. The second electrode is disposed on the first grasping surface. The first electrode and the second electrode are disposed in widthwise directions of the first grasping surface and are disposed in respective positions that are spaced from outer edges of the first grasping surface. The second electrode is disposed on the first grasping surface and the first electrode and the second electrode are disposed in widthwise directions of the first grasping surface, and are disposed in respective positions that are spaced from outer edges of the first grasping surface. When the first grasping surface and the second grasping surface confront one another, a central position between the first electrode and the second electrode is aligned with a central position of the heat generator. The respective first and second grasping surfaces include respective first and second central areas each of which positioned centrally in widthwise directions thereof and confronting one another when they are engaged. At least one of the first grasping surface and the second grasping surface is of a projected shape in which at least one of the first central area and the second central area protrudes toward the other thereof. The first electrode is disposed in the first central area and the second electrode is disposed in one of the first central area and the second central area. The second electrode is disposed on the first grasping surface and the heat generator is disposed on the second grasping jaw.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

### NUMERAL REFERENCE LIST

1 Treatment system
2, 2A to 2C Treatment tool
3 Controller
4 Foot switch
5 Handle
6 Shaft
7, 7A to 7C Grasper
8, 9 Grasping jaw
10, 10A to 10C First electrode
11, 11A to 11C Second electrode
12 Thermal energy applying portion
13 Insulative member
51 Manipulating knob
81, 91 Grasping surface
121 Heat generator
122 Heat transmitter
Ar1, Ar2 First, second central areas
C Electric cable
LT Living tissue
LT1 Treatment target tissue
O1, O2 Central position
R1 Arrow

What is claimed is:

1. A treatment tool comprising:
a first grasping jaw having a first grasping surface;
a second grasping jaw having a second grasping surface and being configured to engage with the first grasping jaw so as to relatively pivot with respect to one another for holding a living tissue therebetween;
a first electrode being disposed on the first grasping surface;
a second electrode being disposed on either the first grasping surface or the second grasping surface and in tandem with the first electrode generates high-frequency energy to the living tissue held therebetween; and
a heat generator disposed in at least one of the first grasping jaw and the second grasping jaw for generating heat when electrically energized,
wherein when the first grasping surface and the second grasping surface confront each other, the first electrode and the second electrode are configured to be disposed in respective positions on both sides of a central position of the heat generator as viewed along directions in which the first grasping surface and the second grasping surface confront each other.

2. The treatment tool of claim 1, wherein when the first grasping surface and the second grasping surface confront one another, the respective first electrode and the second electrode are disposed outside of the heat generator in widthwise directions of the first grasping surface and the second grasping surface as viewed along the directions in which the first grasping surface and the second grasping surface confront each other.

3. The treatment tool of claim 1, wherein the second electrode is disposed on the first grasping surface, and
wherein the first electrode and the second electrode are disposed in widthwise directions of the first grasping surface, and are disposed in respective positions that are spaced from outer edges of the first grasping surface.

4. The treatment tool of claim 2, wherein the second electrode is disposed on the first grasping surface, and
wherein the first electrode and the second electrode are disposed in widthwise directions of the first grasping surface, and are disposed in respective positions that are spaced from outer edges of the first grasping surface.

5. The treatment tool of claim 1, wherein when the first grasping surface and the second grasping surface confront one another, a central position between the first electrode and the second electrode is aligned with a central position of the heat generator.

6. The treatment tool of claim 2, wherein when the first grasping surface and the second grasping surface confront one another, a central position between the first electrode and the second electrode is aligned with a central position of the heat generator.

7. The treatment tool of claim 3, wherein when the first grasping surface and the second grasping surface confront one another, a central position between the first electrode and the second electrode is aligned with a central position of the heat generator.

8. The treatment tool of claim 4, wherein when the first grasping surface and the second grasping surface confront one another, a central position between the first electrode and the second electrode is aligned with a central position of the heat generator.

9. The treatment tool of claim 1, wherein the respective first and second grasping surfaces include respective first and second central areas each of which positioned centrally in widthwise directions thereof and confronting one another when being engaged;
at least one of the first grasping surface and the second grasping surface is of a projected shape in which at least one of the first central area and the second central area protrudes toward the other thereof;
the first electrode is disposed in the first central area; and
the second electrode is disposed in one of the first central area and the second central area.

10. The treatment tool of claim 1, wherein the second electrode is disposed on the first grasping surface; and the heat generator is disposed on the second grasping jaw.

11. The treatment tool of claim 2, wherein the second electrode is disposed on the first grasping surface; and the heat generator is disposed on the second grasping jaw.

12. The treatment tool of claim 3, wherein the second electrode is disposed on the first grasping surface; and the heat generator is disposed on the second grasping jaw.

13. The treatment tool of claim 4, wherein the second electrode is disposed on the first grasping surface; and the heat generator is disposed on the second grasping jaw.

14. The treatment tool of claim 5, wherein the second electrode is disposed on the first grasping surface; and the heat generator is disposed on the second grasping jaw.

15. The treatment tool of claim 1, wherein the second electrode is disposed on the first grasping surface; and the heat generator is disposed on the first grasping jaw.

16. The treatment tool of claim 2, wherein the second electrode is disposed on the first grasping surface; and the heat generator is disposed on the first grasping jaw.

17. The treatment tool of claim 3, wherein the second electrode is disposed on the first grasping surface; and the heat generator is disposed on the first grasping jaw.

18. The treatment tool of claim 4, wherein the second electrode is disposed on the first grasping surface; and the heat generator is disposed on the first grasping jaw.

19. The treatment tool of claim 5, wherein the second electrode is disposed on the first grasping surface; and the heat generator is disposed on the first grasping jaw.
